(19) United States
(12) Patent Application Publication
(10) Pub. No.: US 2024/0355447 A1
(43) Pub. Date: Oct. 24, 2024

(54) METHOD AND APPARATUS FOR DETECTION AND AUTO-INJECTION

(71) Applicant: Resilient Lifescience, Inc., Pittsburgh, PA (US)

(72) Inventors: Maranda Lynn WILLIAMS, Knoxville, TN (US); Charles Robert PROCTOR, Pittsburgh, PA (US); Bradford Alan HOLDEN, Wexford, PA (US)

(73) Assignee: Resilient Lifescience, Inc., Pittsburgh, PA (US)

(21) Appl. No.: 18/686,721

(22) PCT Filed: Aug. 26, 2022
(86) PCT No.: PCT/US2022/075517

§ 371 Date: Feb. 26, 2024

Related U.S. Application Data
(60) Provisional application No. 63/237,440, filed on Aug. 26, 2021.

(51) Int. Cl. G16H 20/17; A61M 5/142; A61M 5/172

(52) U.S. Cl. CPC G16H 20/17; A61M 5/14248; A61M 2005/1726

(57) ABSTRACT

The present system provides a delivery device that may monitor an individual's biometric data. If the biometric data fall below a certain threshold, the delivery device will trigger an alert to medical professionals, indicating the individual's location, as well as alerting friends or other contacts of the individual. In addition, if medical condition is detected, such as an overdose, the delivery device will administer a medicament, such as an opioid antagonist, via intramuscular or subcutaneous injection. The delivery device will continue to administer the medicament until disabled by the individual or a medical professional as long as the delivery device continues to be triggered to do so.

### FIELD OF THE INVENTION

Embodiments relate to a method and apparatus for detection of a medical condition and auto-injection of a medicament.

### BACKGROUND OF THE INVENTION

The United States has been in the midst of an opioid epidemic for decades. The number of individuals that have fatally overdosed on opioids has been increasing steadily. In 2020, over 93,000 individuals fatally overdosed on drugs, and over 70 percent of those involved an opioid. This epidemic is not just an American problem though, it is a global issue. Worldwide, over 16 million people suffer from Opioid Use Disorder (OUD). OUD is a complicated problem. Unlike other substance use disorders, opioid dependence can occur quickly (within 4-8 weeks), cause severe and debilitating symptoms when abruptly stopped, and produce an intense desire to continue using opioids, as the symptoms are so severe.

Complicating this problem even further is the fact that many of the individuals suffering from OUD also suffer from isolation. OUD affects an individual's personal relationships. Those close to the person will begin to distance themselves due to the destructive behavior associated with the disease, and the individual will distance themselves due to the addiction becoming their primary focus.

This isolation has become one of the most dangerous aspects of OUD. Many opioids today are not being manufactured in federally regulated facilities, but instead are being manufactured in illegally operated facilities that include illicit fentanyl in products. The amount of fentanyl and other synthetic opioids found in these illegally manufactured substances often exceed fatal levels. Novel and dangerous analogs of fentanyl are common as well. Fatally overdosing on opioids is a greater risk now than ever before.

There are symptoms associated with a potentially fatal opioid overdose. The preliminary symptoms include drowsiness, slurred speech, slowed movements, and moving in and out of consciousness. These symptoms can appear even while sitting up or in a social situation. During an overdose situation, the person's breathing can become dangerously slow and then stop completely. The heart rate can slow down to a dangerous level as well.

There are pharmaceutical treatments that can be administered during an overdose episode that can increase the chances for survival. These treatments are referred to as “opioid antagonists”. One example of an opioid antagonist is known as naloxone. Naloxone can be administered as a nasal spray (e.g., Narcan) or via injection. Naloxone can reverse the effects of an opioid overdose by blocking opioid receptors and providing a temporary respite from symptoms, creating an opportunity for further medical intervention to prevent a fatality.

A disadvantage of the use of naloxone to treat an overdose is that the individual is often alone. The effect of an opioid can impact motor control. The individual may be unconscious during the overdose. In addition, the naloxone must be administered correctly in the right amount and manner, and further medical intervention is often required. If an overdosing individual is alone, it may be quite difficult for them to realize they are in a dangerous situation, or to effectively administer the naloxone and request medical help by themselves.

Accordingly, there is a need for an apparatus that detects a medical condition, such as an overdose condition, and auto-injects a medicament, such as an opioid antagonist, into an individual.

### SUMMARY OF THE INVENTION

Embodiments relate to a delivery device that can monitor an individual's biometric data (e.g., heart rate and blood oxygen levels). If the levels of the biometric data fall below a certain threshold, the delivery device may trigger an alert to medical professionals, indicating the individual's location, as well as alerting friends or other contacts of the individual. In addition, if a medical condition is detected, the system will administer a medicament via injection. The delivery device will continue to administer the medicament until disabled by a medical professional as long as the delivery device continues to be triggered to do so.

In an exemplary embodiment, the delivery device, comprises a housing, a patch comprising a first surface and a second surface, wherein the first surface is coupled to the housing, a processing module positioned within the housing, and an administration module positioned within the housing. The processing module comprises a monitoring module configured to monitor an individual's biometric data, and a sensing module configured to collect the individual's biometric data. The administration module positioned comprises a reservoir configured to store a medicament, a cannula configured to transport the medicament from the reservoir to an insertion mechanism, an insertion mechanism configured to pierce an individual's skin and establish a pathway for delivery of the medicament from the cannula to the individual, and a drive mechanism configured to release the medicament from the reservoir and to deliver the medicament through the cannula and the insertion mechanism to the individual.

In some embodiments, the insertion mechanism comprises a needle, wherein the needle is configured to pierce the individual's skin and deliver the medicament.

In some embodiments, the insertion mechanism comprises the cannula and an insertion member configured to pierce the individual's skin and place the cannula within the individual's skin, wherein the insertion member is a needle or a trocar.

In some embodiments, the second surface of the patch is attached to the individual.

In some embodiments, the patch is a thin fabric adhesive.

In some embodiments, the second surface of the patch is attached to the individual's abdomen, chest, calf, upper leg, or upper arm.

In some embodiments, the sensing module comprises a PPG sensor.

In some embodiments, the sensing module comprises an accelerometer.

In some embodiments, the sensing module comprises a temperature sensor.

In some embodiments, the medicament is an opioid antagonist.

In some embodiments, the opioid antagonist comprises naloxone, naltrexone, or nalmefene.

In some embodiments, the medicament is a partial opioid antagonist.

In some embodiments, the partial opioid antagonist comprises buprenorphine.

In some embodiments, the delivery device further comprises a trigger injection button positioned on an external surface of the housing.

In some embodiments, the trigger injection button is configured to actuate the drive mechanism of the administration module.

In some embodiments, the monitoring module is further configured to detect an overdose based on the individual's biometric data.

In some embodiments, the processing module further comprises an alarm module in communication with the processing module, wherein the alarm module is configured to trigger an alarm upon the monitoring module detecting an overdose.

In some embodiments, the alarm is a vibratory signal.

In some embodiments, the alarm is an auditory signal.

In some embodiments, the alarm is an optical signal.

In some embodiments, the delivery device further comprises a delay injection button positioned on an external surface of the housing.

In some embodiments, the delay injection button is configured to deactivate the alarm triggered by the alarm module.

In some embodiments, the alarm module is configured to actuate the drive mechanism of the administration module if the delay injection button is not actuated.

In some embodiments, the monitoring module comprises an algorithm configured to monitor changes in the individual's biometric data.

In some embodiments, the drive mechanism may be actuated or delayed based on the changes in the individual's biometric data.

In some embodiments, the drive mechanism is configured to continuously deliver the medicament to the individual.

In an exemplary embodiment, a method of detecting an overdose condition in an individual comprises detecting an overdose using a delivery device, alerting medical authorities; and providing a medicament to the individual.

In some embodiments, the delivery device comprises a housing, a patch comprising a first surface and a second surface, wherein the first surface is coupled to the housing, a processing module positioned within the housing, and an administration module positioned within the housing. The processing module comprises a monitoring module configured to monitor an individual's biometric data and to detect an overdose based on the individual's biometric data, and a sensing module configured to collect the individual's biometric data. The administration module comprises a reservoir configured to store a medicament, a cannula configured to transport the medicament from the reservoir to an insertion mechanism, an insertion mechanism configured to pierce an individual's skin and establish a pathway for delivery of the medicament from the cannula to the individual, and a drive mechanism configured to release the medicament from the reservoir and to drive the medicament through the cannula and the insertion mechanism to the individual.

In some embodiments, the processing module further comprises an alarm module in communication with the processing module, wherein the alarm module is configured to trigger an alarm upon the monitoring module detecting an overdose and to perform the step of alerting medical authorities.

In some embodiments, the insertion mechanism comprises a needle and the step of providing a medicament to the individual comprises the needle piercing the individual's skin, and the needle delivering the medicament.

In some embodiments, the insertion mechanism comprises the cannula and an insertion member configured to pierce the individual's skin and place the cannula within the individual's skin and the step of providing a medicament to the individual comprises the cannula and the insertion member piercing the individual's skin; removing the insertion member from the individual's skin and leaving the cannula in the individual's skin; and the cannula delivering the medicament, wherein the insertion member is a needle or a trocar.

In some embodiments, the method further comprises continuing to provide the medicament to the individual until the delivery device is disabled.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is a functional block diagram of an exemplary embodiment of a delivery device.

FIG. 2 is a flow diagram illustrating operation of an exemplary embodiment of a delivery device.

FIG. 3 is a perspective view of an exemplary embodiment of a delivery device.

FIG. 4 is a perspective view of an exemplary embodiment of a delivery device.

FIG. 5 is an exploded view of an exemplary embodiment of a delivery device.

FIG. 6 shows internal components of an exemplary embodiment of a delivery device.

FIG. 7 shows internal components of an exemplary embodiment of a delivery device.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of various aspects of the present invention. The scope of the present invention is not limited by this description.

The system is a method and apparatus for detecting a medical condition and administering a medicament to an individual while alerting medical professionals and emergency services.

From the perspective of an overdose condition, the use of an opioid antagonist can be effective in temporary amelioration of a drug overdose episode. The problems with the current use of an antagonist to an opioid overdose are 1) the need for someone to be available at the time of the overdose to administer the antagonist; 2) the need for the dose to be administered before cerebral hypoxia occurs; 3) the possible need for multiple doses of the antagonist to counter the effects of the opioids; and 4) the need for emergency contacts and medical personnel to know that the overdose has occurred and where to find the individual.

The present apparatus is a multifunction delivery device that may be attached to the individual's body for extended periods of time, via an interchangeable adhesive, a strap, or some other suitable means. The device may perform the functions monitoring/detecting a medical condition; administering of a medicament; and alerting of medical professionals.

### Apparatus—Delivery Device

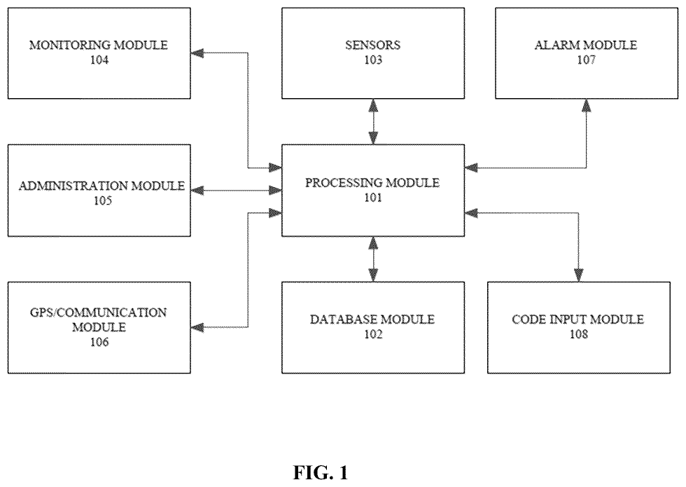

Referring to FIG. 1, the delivery device is controlled by a processing module 101 that interacts with, and controls, the other modules. The monitoring module 104 will monitor one or more of the individual's respiratory rate, ventilation, heart rate, carbon dioxide levels, and blood oxygen levels through sensors 103 that will be in contact with the person's skin. Baseline information for the individual's blood oxygen level and heart rate are stored in database module 102.

If the individual's levels fall below the individual's predetermined set parameters, such as a heart rate below 60 beats per minute and a blood oxygen level below 90%, the device will trigger an alarm via alarm module 107. It is contemplated that the alarm may be a vibratory, auditory, optical, or any other suitable signal or combinations thereof to alert the individual. Once the alarm has been triggered, the device will simultaneously begin locating the individual's GPS location using the GPS/communication module 106. In an exemplary embodiment, the module 106 may constantly be tracking the location of the individual so that the information is more readily available in an emergency situation.

When the alarm goes off, the individual can deactivate the alarm by pressing a button or entering a code in code input module 108. This may be an alpha-numeric keypad that is physical or virtual, and may be presented as a touch screen. For example, the individual may have to deactivate the alarm within a certain time period (e.g., fifteen seconds) to prevent the delivery device from moving forward in the algorithm. In an exemplary embodiment, deactivating the alarm will require knowing a passcode (e.g., gesture, multi-digit PIN, biometric key, phrase, or any other suitable passcode), therefore preventing accidental deactivation by the individual and improper deactivation by anyone else that might be nearby the individual. An assumption is that the individual may have an idea if they are in danger or not, and the ability to deactivate the alarm is an indication of alertness that may indicate that a medical condition (i.e., an overdose condition) is not taking place.

In an exemplary embodiment, even if the individual manages to deactivate the device, the device will still continue to monitor their heart rate and oxygen levels to ensure that the device did not in fact detect a medical condition that it was prematurely prevented from inhibiting. If the device is not deactivated, the individual's GPS coordinates will be sent to local emergency services via GPS/communication Module 106. The GPS/communication module 106 may communicate via WiFi, cellular connections, Bluetooth, radio, or any other suitable communication methods. Along with the coordinates the system will indicate a possible overdose, with the individual's name and a time range of data from the monitoring module 104 (e.g., a 15 minute block of data) so that the medical professionals can be fully prepared when they arrive on the scene.

Alarm module 107 may also send an auto-generated text message to the individual's emergency contacts that are stored in database module 102, informing the contacts that a suspected medical condition (e.g., an overdose) has occurred and that emergency services have been contacted. In an exemplary embodiment, the contacts are informed of the individual's location so that they might be able to help sooner than emergency services.

When the alarm is triggered and not deactivated, the administration module 105 will administer a measured dose of the medicament to the individual. In exemplary embodiments, the medicament comprises an opioid antagonist or a partial opioid antagonist. In preferred embodiments, the opioid antagonist comprises naloxone, naltrexone, nalmefene, or any other suitable opioid antagonist or mixtures thereof. In preferred embodiments, the partial opioid antagonist comprises buprenorphine. In an exemplary embodiment, the administration module 105 can maintain the medicament at a desired temperature range (e.g., between 59° F. and 86° F.), to maintain efficacy of the medicament for eventual use. In one embodiment, the medicament will be administered intramuscularly, and the administration module 105 will include a needle capable of doing as such. In another embodiment, the medicament will be administered subcutaneously, and the administration module 105 will include a needle or trocar coupled with a cannula, where the needle or trocar pierces the individual's skin, places the cannula, and then retracts leaving the cannula in place to deliver the medicament. The administration module 105 will hold multiple doses of the medicament and be capable of injecting a set number of milligrams of dosage of the medicament each time the device determines the need. The medicament will enter the central nervous system post-administration. Depending on the dose and medical condition, the individual may not see full reversal of the effects without the additional doses of medicament that will follow. The administration module 105 may be refillable and reusable.

The delivery device may be a single delivery device or several devices in communication with each other. In one embodiment, the monitoring module 104, alarm module 107, sensors 103, code input module 108, and GPS/communication module 106 are in a first device while the administration module 105 is in a second device that is in communication with the first device. The first device and the second device may be placed on any body part of an individual. In a preferred version of this embodiment, the first device may be placed on an individual's chest or abdomen, and the second device may be placed on an individual's upper arm, thigh, or calf to establish a location for intramuscular injection. In a preferred embodiment, the delivery device is a single device, containing all modules. The device may be placed anywhere on the body. In a preferred embodiment, the device is placed on the user's abdomen, establishing a site for subcutaneous injection.

The delivery device will need to be able to work without a cellular phone, but it will also have the capability to work with the individual's cellular phone via an application if the individual owns a cellular phone, thus the devices have wireless communication capabilities. In an exemplary embodiment, an application on the individual's phone will keep a log of the individual's heart rate and oxygen levels so that they can better understand how the medical condition is affecting them, and so they can also share these results with their medical provider(s). The individual's log will allow the medical provider to make adjustments to the individual's algorithm as needed, allowing the device to be more sensitive if near-miss overdoses are detected by the provider. The log and algorithm will have the capabilities to potentially predict when an overdose is about to occur, further decreasing the amount of time between a future overdose and administration of the medicament and contacting of emergency services. The application will also be how the individual can upload the information for their selected emergency contacts that will receive the auto-generated text should a medical condition (e.g., an overdose) occur.

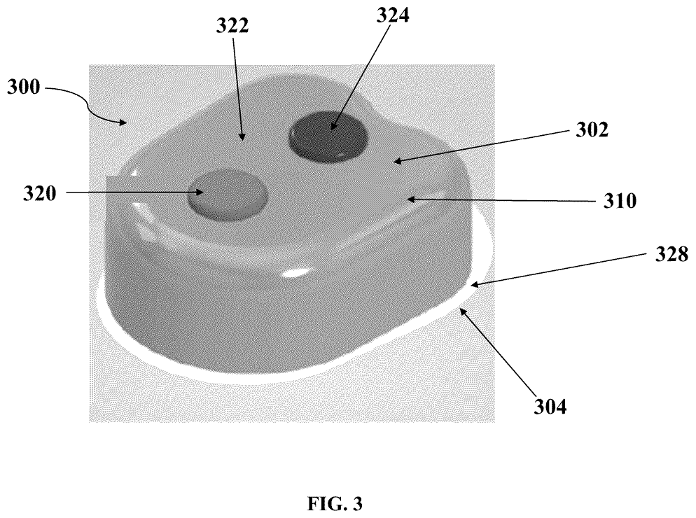

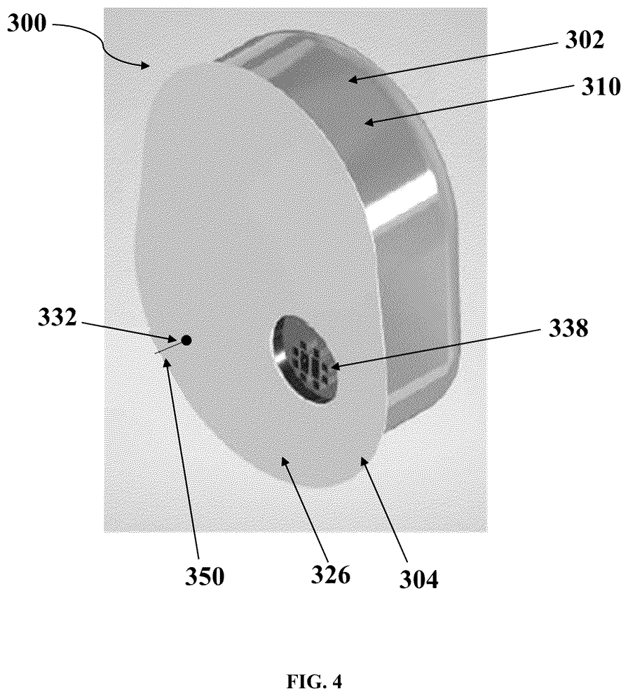

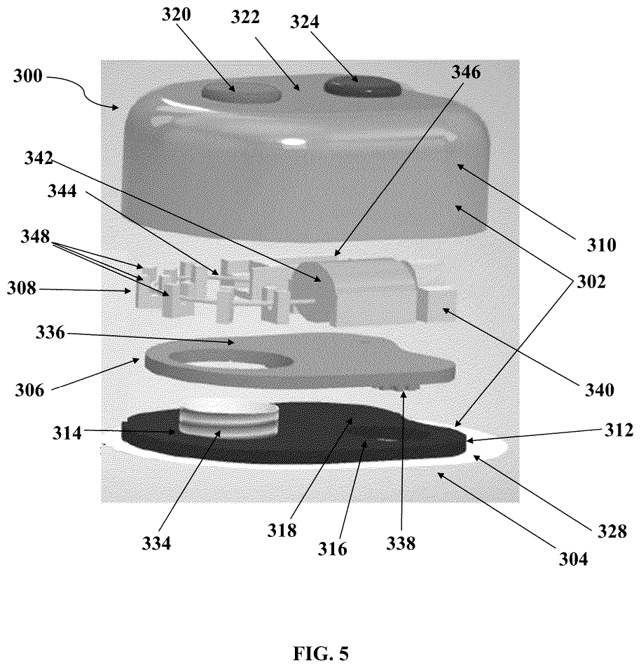

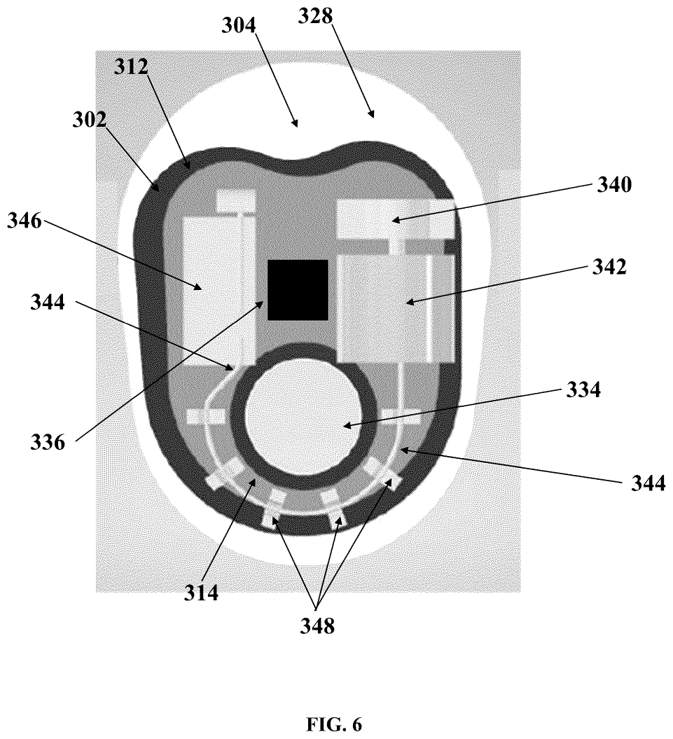

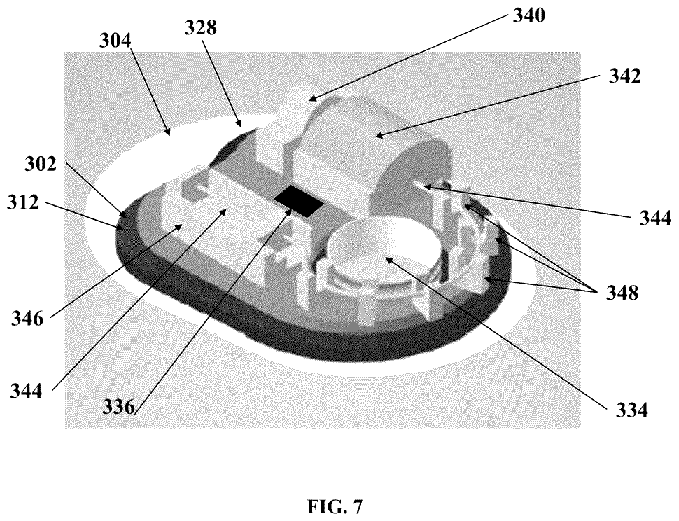

Referring now to FIGS. 3-7, an embodiment of an exemplary delivery device as described with regard to FIG. 1 is provided. In this embodiment, a delivery device 300 comprises a housing 302, a patch 304 couplable to the housing 302, a processing module 306 positioned within the housing 302, and an administration module 308 positioned within the housing 302.

The housing 302 may comprise a first portion 310 and a second portion 312, wherein the first portion 310 is couplable to the second portion 312 such that the first portion 310 and the second portion 312 form an encapsulated region. It is understood that encapsulated means enclosed (e.g., surrounded on all sides). It is contemplated that the first portion 310 may be coupled to the second portion 312 (by threading, a snap connection, a press fit connection, or any other suitable coupling). It is further contemplated that the first portion 310 may be removably coupled to the second portion 312.

The second portion 312 may be of any shape, but it is contemplated that the second portion 312 may be flat or substantially flat. It is contemplated that the second portion 312 may comprise a first bore 314, a second bore 316, and a needle bore 318. The first portion 310 may be of any shape, but it is contemplated that the first portion 310 may be a three-dimensional shape such that when the first portion 310 is coupled to the second portion 312, the three-dimensional properties of the first portion 310 allow for the encapsulated region.

The housing 302 may be made of a protective material (e.g., hard plastic-like material or any other suitable material). It is further contemplated that the housing 302 may be made of a waterproof and/or water resistant material.

The delivery device 300 may further comprise a trigger injection button 320. The trigger injection button 320 may be positioned on an external surface 322 of the first portion 310 of the housing 302, such that the trigger injection button 320 may be visible to and interacted with (e.g., pressed) by an individual. The trigger injection button 320 may be configured to actuate the administration module 308. The trigger injection button 320 may be a mechanical button or a touch-activated surface. For example, an individual may press/touch the trigger injection button 320 to actuate the administration module 308.

The delivery device may further comprise a delay injection button 324. The delay injection button 324 may be located on an external surface 322 of the housing 302, such that the delay injection button 324 may be visible to and interacted with (e.g., pressed) by an individual. The delay injection button 324 may be configured to delay actuation of the administration module 308. The delay injection button 324 may be a mechanical button or a touch-activated surface. For example, an individual may press/touch the delay injection button 324 to delay actuation of the administration module 308.

In exemplary embodiments, the trigger injection button 320 and the delay injection button 324 may comprise distinctive properties (e.g., different shapes, different colors, or any other suitable distinctive property) such that an individual may easily and readily differentiate between each button.

In exemplary embodiments, the patch 304 may be a thin layer of material. In a preferred embodiment, the patch 304 may be a thin fabric adhesive. The patch 304 may comprise a first surface 326 and a second surface 328. The first surface 326 of the patch 304 may be configured to attach to a surface (e.g., skin), such that the patch 304 may achieve intimate contact with the surface. For example, the patch 304 may be applied directly on skin and achieve intimate contact with the skin for efficient injection and administration. It is contemplated that the patch 304 may be applied to an individual's abdomen, chest, calf, upper leg, upper arm, or any other suitable body part. It is contemplated that the first surface 326 may attach to the surface via adhesives. The patch 304 may further comprise a first bore 330, wherein the first bore 330 aligns with the second bore 316 of the second portion 312 of the housing 302, and a needle bore 332, wherein the needle bore 332 aligns with the needle bore 318 of the second portion 312 of the housing 302.

The second surface 328 of the patch 304 may be configured to be couplable to the second portion 312 of the housing 302. It is contemplated that the second surface 328 of the patch 304 may be coupled to the second portion 312 of the housing 302 (by threading, a snap connection, a press fit connection, adhesives, or any other suitable coupling). It is further contemplated that the second surface 328 of the patch 304 may be removably coupled to the second portion 312 of the housing 302. For instance, a protruding member 334 may be positioned on the second surface 328 of the patch 304 and be configured to align with the first bore 314 of the second portion 312 of the housing 302 such that the protruding member 334 may be secured within the first bore 314 of the second portion 312 of the housing 302 (by threading, a snap connection, a press fit connection, adhesives, or any other suitable coupling).

The processing module 306 may abut the second portion 312 of the housing 302 such that the processing module 306 is positioned within the encapsulated region (e.g., the space formed by the first portion 310 and second portion 312 of the housing 302). The processing module 306 is in communication with one or more additional modules, including but not limited to a monitoring module 336, a sensing module 338, a GPS/communication module (not shown), a database module (not shown), an alarm module (not shown), and a code input module (not shown).

In exemplary embodiments, a monitoring module 336 may be in communication with the processing module 306. It is contemplated that the monitoring module 336 may be electrically connected to the processing module 306. The monitoring module 336 may be configured to monitor an individual's biometric data (e.g., heart rate, respiratory rate, ventilation, carbon dioxide levels, blood oxygen levels, or any other suitable biometric data). It is contemplated that an individual's biometric data may be collected by a sensing module 338.

In exemplary embodiments, a database module may be in communication with the processing module 306. It is contemplated that the database module may be electrically connected to the processing module 306. It is further contemplated that the database module may have no physical connection to the processing module 306, and may be positioned outside of the housing 302. Information (e.g., predetermined set parameters) regarding the individual's biometric data may be stored in the database module such that, if an individual's biometric data falls below the individual's predetermined set parameters stored in the database module, an alarm module may be actuated. For example, if an individual's heart rate falls below 60 beats per minute or if an individual's blood oxygen level falls below 90%, the alarm module may be actuated.

In exemplary embodiments, an alarm module may be in communication with the processing module 306. It is contemplated that the alarm module may be electrically connected to the processing module 306. It is further contemplated that the alarm module may have no physical connection to the processing module 306, and may be positioned outside of the housing 302. It is contemplated that actuation of the alarm module triggers an alarm. It is contemplated that the alarm may be a vibratory, auditory, optical, or any other suitable signal or combinations thereof to alert the individual. It is further contemplated that once the alarm module is actuated, the GPS/communication module may be simultaneously actuated.

In exemplary embodiments, a GPS/communication module may be in communication with the processing module 306. It is contemplated that the GPS/communication module may be electrically connected to the processing module 306. It is further contemplated that the GPS/communication module may have no physical connection to the processing module 306, and may be positioned outside of the housing 302. The GPS/communication module may be configured to track the location information of the individual so that the location information is more readily available in an emergency situation. The GPS/communication module may communicate individual information with third-party devices via WiFi, cellular connections, Bluetooth, radio, etc., including but not limited to the individual's name, location, a time range of data from the monitoring module 336, or any other suitable information. The GPS/communication module may allow medical professionals to be fully prepared when they arrive on the scene.

It is contemplated that, when the alarm module is actuated, an individual may deactivate the alarm by pressing a button (e.g., the delay injection button 324) or entering a code in a code input module. The code input module may be in communication with the processing module. It is contemplated that the code input module may be electrically connected to the processing module 306. It is further contemplated that the code input module may have no physical connection to the processing module 306, and may be positioned outside of the housing 302. For instance, the code input module may be an alpha-numeric keypad that is physical or virtual, and may be presented as a touch screen. Deactivation of the alarm module by using the code input module is an indication of alertness that may indicate that an overdose is not taking place.

It is contemplated that the alarm module may be configured to send an auto generated text message to the individual's emergency contacts stored in the database module, informing the contacts that a suspected overdose has occurred and that emergency services have been contacted.

The sensing module 338 may be electrically connected to the processing module 306. The sensing module 338 is configured to be in contact with an individual's skin, such that the sensing module 338 may efficiently and accurately collect an individual's vital signs. For instance, the sensing module 338 may align with the first bore 330 of the patch 304 and the second bore 316 of the second portion 312 of the housing 302 such that the sensing module 338 may be exposed to and make contact with an individual's skin. It is contemplated that the sensing module 338 may comprise a photoplethysmography (PPG) sensor, an accelerometer, a temperature sensor, or any combination of the above.

The administration module 308 may abut the processing module 306 such that the administration module 308 is positioned within the encapsulated region (e.g., the space formed by the first portion 310 and second portion 312 of the housing 302). The administration module 308 may comprise a drive mechanism 340, a reservoir 342, a cannula 344, and an insertion mechanism 346.

The drive mechanism 340 may be configured to release an amount of medicament from the reservoir 342. It is contemplated that the amount of medicament to be released may be predetermined and/or preset. Alternatively, the amount of medicament to be released may vary (e.g., increase the rate of medicament released or decrease the amount of medicament released) based on the change in the individual's biometric data as monitored by the monitoring module 336. It is contemplated that the drive mechanism 340 may be actuated upon actuation of the trigger injection button 320. In an alternative embodiment, when the alarm module is triggered and not deactivated, the drive mechanism will be actuated 340.

It is contemplated that when the alarm module is triggered and the delay injection button is actuated, the drive mechanism 340 will not be actuated.

The reservoir 342 may be configured to store the medicament. In exemplary embodiments, the medicament comprises an opioid antagonist or a partial opioid antagonist. In preferred embodiments, the opioid antagonist comprises naloxone, naltrexone, nalmefene, or any other suitable opioid antagonist or mixtures thereof. In preferred embodiments, the partial opioid antagonist comprises buprenorphine. It is contemplated that the reservoir 342 may maintain the medicament at a desired temperature range (e.g., between 59° F. and 86° F.), to maintain efficacy of the medicament for eventual use.

In exemplary embodiments, upon actuation of the drive mechanism 340, the reservoir 342 releases the medicament to a cannula 344, and the cannula 344 is configured to transport the medicament to the insertion mechanism 346. It is contemplated that the cannula 344 is supported by a supporting means 348 (e.g., guides, tracks, or any other suitable supporting means).

In exemplary embodiments, the insertion mechanism 346 is configured to inject a needle 350 into an individual's skin. The needle 350 may be positioned within the aligned needle bores 318 and 332 of the second portion 312 of the housing 302 and the patch 304. In operation and upon actuation of the insertion mechanism 346, the needle 350 may be inserted into an individual's skin.

In an exemplary embodiment, an insertion member (e.g., the needle 350 or a trocar) and a cannula may be inserted into an individual together, the insertion member is removed and the cannula is left behind, and the drive mechanism 340 releases the medicament from the reservoir 342 and delivers the medicament through the cannula, to the insertion mechanism 346, and into an individual. It is contemplated that the cannula left in the individual may be the same cannula 344 that is configured to transport the medicament from the reservoir 342 to the insertion mechanism 346, or it may be a different cannula.

In exemplary embodiments, the monitoring module may comprise an algorithm configured to monitor changes in the individual's biometric data. The drive mechanism may be actuated or delayed based on the changes in the individual's biometric data. For instance, the drive mechanism may increase or decrease the rate of medicament injection based on the changes in the individual's biometric data.

### Method and Use

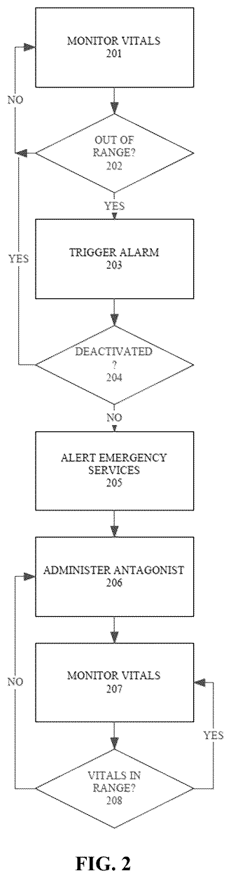

Referring to FIG. 2, at step 201 the system monitors the vitals of an individual. In an exemplary embodiment, the vitals are blood oxygen level and heart rate. At decision block 202 it is determined if the vitals are out of a desired range. If not, they system returns to step 201 and continues monitoring the individual.

If the vitals are out of range at step 202 the system triggers an alarm at step 203. At decision block 204 it is determined if the alarm has been deactivated. If so, the system returns to step 201 to monitor the individual. In an exemplary embodiment, the alarm has not been deactivated at step 204 the system alerts emergency services at step 205. This includes providing GPS information and other individual information to emergency services.

At step 206 the system administers a medicament to the individual. At step 207 the system monitors the vitals of the individual after the medicament has been injected. At decision block 208 it is determined if the vitals are in range. If so, the system continues to monitor at step 207. If not, the system administers another dose of the medicament at step 206.

### EXAMPLES

Example 1: An adult weighing 80 kg attached a patch. The individual attaches the device to their abdomen such that a PPG sensor and injection site sit between 2 cm and 15 cm to either side of center. The individual then consumes enough of an opioid to induce respiratory depression; onset of respiratory depression occurs 10 minutes after consumption. The device detects said respiratory depression and sounds an alarm. The alarm continues for 15 seconds without the individual deactivating the alarm, so the device triggers the antagonist administration module. The device inserts a cannula into the individual's subcutaneous tissue and administers the opioid antagonist. The opioid antagonist from the initial administration is sufficient to reverse the respiratory depression, and the individual's ventilation returns to baseline 90 seconds post-administration. The device ceases to deliver the drug antagonist.

Example 2: An adult weighing 80 kg attaches a patch to the delivery device. They attach the device to their abdomen such that the PPG sensor and injection site sit between 2 cm and 15 cm to either side of center. The individual then consumes enough of an opioid to induce respiratory depression; onset of respiratory depression occurs 10 minutes after consumption. The device detects said respiratory depression and sounds an alarm. The alarm continues for 15 seconds without the individual deactivating the alarm, so the device triggers the antagonist administration module. The device inserts a cannula into the individual's subcutaneous tissue and administers the opioid antagonist. The opioid antagonist from the initial administration is not sufficient to reverse the respiratory depression within 90 seconds. The device delivers an additional dose of the opioid antagonist, which is sufficient to reverse the respiratory depression. The individual's ventilation returns to baseline 90 seconds after the second administration. The device ceases to deliver the drug antagonist.

Example 3: An adult weighing 80 kg attached a patch to the delivery device. They attach the device to their abdomen such that the PPG sensor and injection site sit between 2 cm and 15 cm to either side of center. The individual does not consume any opioids. The individual practices depressed ventilation during meditation. The device detects said respiratory depression and sounds an alarm. The individual disables the alarm. The device ceases the alarm, does not administer any opioid antagonist, and continues to monitor the individual's respiration.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of the apparatus and methods of making and using the same have been discussed and illustrated herein, it is to be distinctly understood that the invention is not limited thereto but may be otherwise variously embodied and practiced within the scope of the following claims.

What is claimed is:

1. A delivery device, comprising:
a housing;
a patch comprising a first surface and a second surface, wherein the first surface is coupled to the housing;
a processing module positioned within the housing, wherein the processing module comprises:
a monitoring module configured to monitor an individual's biometric data, and
a sensing module configured to collect the individual's biometric data; and
an administration module positioned within the housing, wherein the administration module comprises:
a reservoir configured to store a medicament,
a cannula configured to transport the medicament from the reservoir to an insertion mechanism,
an insertion mechanism configured to pierce an individual's skin and establish a pathway for delivery of the medicament from the cannula to the individual, and
a drive mechanism configured to release the medicament from the reservoir and deliver the medicament through the cannula and the insertion mechanism to the individual.

2. The delivery device of claim 1, wherein the insertion mechanism comprises a needle, wherein the needle is configured to pierce the individual's skin and deliver the medicament.

3. The delivery device of claim 1, wherein the insertion mechanism comprises
a cannula; and
an insertion member configured to pierce the individual's skin and place the cannula within the individual's skin, wherein the insertion member is a needle or a trocar.

4. The delivery device of claim 1, wherein the second surface of the patch is attached to the individual.

5. The delivery device of claim 4, wherein the patch is a thin fabric adhesive.

6. The delivery device of claim 4, wherein the second surface of the patch is attached to the individual's abdomen, chest, calf, upper leg, or upper arm.

7. The delivery device of claim 1, wherein the sensing module comprises a PPG sensor.

8. The delivery device of claim 1, wherein the sensing module comprises an accelerometer.

9. The delivery device of claim 1, wherein the sensing module comprises a temperature sensor.

10. The delivery device of claim 1, wherein the medicament is an opioid antagonist.

11. The delivery device of claim 10, wherein the opioid antagonist comprises naloxone, naltrexone, or nalmefene.

12. The delivery device of claim 1, wherein the medicament is a partial opioid antagonist.

13. The delivery device of claim 12, wherein the partial opioid antagonist comprises buprenorphine.

14. The delivery device of claim 1, further comprising a trigger injection button positioned on an external surface of the housing.

15. The delivery device of claim 14, wherein the trigger injection button is configured to actuate the drive mechanism of the administration module.

16. The delivery device of claim 1, wherein the monitoring module is further configured to detect an overdose based on the individual's biometric data.

17. The delivery device of claim 16, wherein the processing module further comprises an alarm module in communication with the processing module, wherein the alarm module is configured to trigger an alarm upon the monitoring module detecting an overdose.

18. The delivery device of claim 17, wherein the alarm is a vibratory signal.

19. The delivery device of claim 17, wherein the alarm is an auditory signal.

20. The delivery device of claim 17, wherein the alarm is an optical signal.

21. The delivery device of claim 17, further comprising a delay injection button positioned on an external surface of the housing.

22. The delivery device of claim 21, wherein the delay injection button is configured to deactivate the alarm triggered by the alarm module.

23. The delivery device of claim 22, wherein the alarm module is configured to actuate the drive mechanism of the administration module if the delay injection button is not actuated.

24. The delivery device of claim 1, wherein the monitoring module comprises an algorithm configured to monitor changes in the individual's biometric data.

25. The delivery device of claim 24, wherein the drive mechanism may be actuated or delayed based on the changes in the individual's biometric data.

26. The delivery device of claim 25, wherein the drive mechanism is configured to continuously deliver the medicament to the individual.

27. A method of detecting an overdose condition in an individual, comprising:
detecting an overdose using a delivery device;
alerting medical authorities of the overdose; and
providing a medicament to the individual.

28. The method of claim 27, wherein the delivery device comprises:
a housing;
a patch comprising a first surface and a second surface, wherein the first surface is coupled to the housing;
a processing module positioned within the housing, wherein the processing module comprises:
a monitoring module configured to monitor an individual's biometric data and to detect an overdose based on the individual's biometric data, and
a sensing module configured to collect the individual's biometric data; and
an administration module positioned within the housing, wherein the administration module comprises:
a reservoir configured to store a medicament,
a cannula configured to transport the medicament from the reservoir to an insertion mechanism,
an insertion mechanism configured to pierce an individual's skin and establish a pathway for delivery of the medicament from the cannula to the individual, and
a drive mechanism configured to release the medicament from the reservoir and to drive the medicament through the cannula and the insertion mechanism to the individual.

29. The method of claim 28, wherein the processing module further comprises an alarm module in communication with the processing module and wherein the alarm module is configured to trigger an alarm upon the monitoring module detecting an overdose, and the step of alerting medical authorities of the overdose comprises:
alerting medical authorities via the alarm module.

30. The method of claim 28, wherein the insertion mechanism comprises a needle, the step of providing a medicament to the individual comprises:
the needle piercing the individual's skin; and
the needle delivering the medicament.

31. The method of claim 28, wherein the insertion mechanism comprises the cannula and an insertion member configured to pierce the individual's skin and place the cannula within the individual's skin, the step of providing a medicament to the individual comprises:
the cannula and the insertion member piercing the individual's skin;
removing the insertion member from the individual's skin and leaving the cannula in the individual's skin; and
the cannula delivering the medicament,
wherein the insertion member is a needle or a trocar.

32. The method of claim 27, further comprising:
continuing to provide the medicament to the individual until the delivery device is disabled.
